(19) United States
(12) Patent Application Publication
(10) Pub. No.: US 2017/0086718 A1
(43) Pub. Date: Mar. 30, 2017

(54) Methods and Apparatuses for Providing Adverse Condition Notification in Analyte Monitoring Systems

(71) Applicant: Abbott Diabetes Care Inc., Alameda, CA (US)

(72) Inventors: Jean-Pierre Cole, Tracy, CA (US); Namvar Kiaie, Danville, CA (US)

(73) Assignee: Abbott Diabetes Care Inc., Alameda, CA (US)

(21) Appl. No.: 15/377,985

(22) Filed: Dec. 13, 2016

Related U.S. Application Data
(63) Continuation of application No. 14/028,372, filed on Sep. 16, 2013.
(60) Provisional application No. 61/702,227, filed on Sep. 17, 2012.

(51) Int. Cl. A61B 5/145; A61B 5/00

(52) U.S. Cl. CPC A61B 5/14532; A61B 5/14542; A61B 5/6813; A61B 5/7203; A61B 5/7275; A61B 2560/0271

(57) ABSTRACT

Methods, devices, systems, and kits are provided that buffer the time spaced glucose signals in a memory, and when a request for real time glucose level information is detected, transmit the buffered glucose signals and real time monitored glucose level information to a remotely located device, process a subset of the received glucose signals to identify a predetermined number of consecutive glucose data points indicating an adverse condition such as an impending hypoglycemic condition, confirm the adverse condition based on comparison of the predetermined number of consecutive glucose data points to a stored glucose data profile associated with the adverse condition, where confirming the adverse condition includes generating a notification signal when the impending hypoglycemic condition is confirmed, and activate a radio frequency (RF) communication module to wirelessly transmit the generated notification signal to the remotely located device only when the notification signal is generated.

### RELATED APPLICATIONS

The present application is a continuation of U.S. patent application Ser. No. 14/028,372 filed Sep. 16, 2013, which claims priority to U.S. Provisional Application No. 61/702,227 filed Sep. 17, 2012, entitled “Methods and Apparatuses for Providing Adverse Condition Notification in Analyte Monitoring Systems”, the disclosure of which is incorporated herein by reference for all purposes.

### BACKGROUND

The detection of the concentration level of glucose or other analytes in certain individuals is vitally important to their health. For example, the monitoring of glucose levels is particularly important to individuals with diabetes or pre-diabetes. People with diabetes may need to monitor their glucose levels to properly control their glycemic levels.

Devices such as sensors have been developed for continuous and automatic in vivo monitoring of analyte characteristics, such as glucose levels, in bodily fluids such as in the blood stream or in interstitial fluid. Some of these analyte measuring sensors are configured so that at least a portion of the devices are positioned below a skin surface of a user, e.g., in a blood vessel or in the subcutaneous tissue of a user. Information obtained from such devices can provide real time analyte levels which can indicate detected levels that require immediate attention, including intervention. It would be desirable to have an in vivo analyte monitoring system which provides warnings or notifications of onset of adverse physiological conditions detected by the analyte monitoring system such as hypoglycemic conditions.

### SUMMARY

Provided herein are methods, apparatuses, systems and kits for in vivo monitoring of analyte levels that include, for example, wearable on body sensor electronics operatively coupled to a transcutaneously positioned in vivo analyte sensor for real time in vivo monitoring of analyte levels, where the sensor electronics includes programming to detect and generate adverse condition notification such as detection of hypoglycemic condition, hyperglycemic conditions, rapidly changing glucose levels, or other adverse medical conditions in the case of a glucose monitoring system, based on which, the sensor electronics activates its radio frequency (RF) communication module to transmit the generated notification to a user interface device such as a reader device or other electronic data communication/processing devices that is remote to the sensor electronics, to notify the user of the detected adverse condition. In all other instances, sensor electronics communication lays dormant and only transmits sensed information when it receives a signal from its external receiving device, as determined by a user. In this manner, critical sensing information is identified and communicated immediately, while all other sensing information is only communicated when a user wants it.

In some embodiments, methods are implemented using one or more computer processors. The methods include receiving time spaced glucose signals from an in vivo positioned glucose sensor in fluid contact with interstitial fluid (or other bodily fluid), buffering the received time spaced glucose signals in a memory, detecting a request for real time glucose level information, where when the request for real time glucose level information is detected, transmitting the buffered glucose signals and/or real time glucose signal received from the glucose sensor to a remotely located device using a first or non hypoglycemic communication protocol such as backscattering radio wave, processing a subset of the received time spaced glucose signals to identify a predetermined number of consecutive glucose data points from the subset of the received time spaced glucose signals indicating a hypoglycemic condition or an impending hypoglycemic condition, confirming the hypoglycemic condition or impending hypoglycemic condition based on comparison of the predetermined number of consecutive glucose data points to a stored glucose data profile associated with the impending hypoglycemic condition, where confirming the impending hypoglycemic condition includes generating a notification signal when the impending hypoglycemic condition is confirmed, and activating a second communication protocol or hypoglycemic communication protocol such as a radio frequency (RF) communication module to wirelessly transmit the generated notification signal to the remotely located device only when the notification signal is generated.

In some other embodiments, apparatus includes a user interface, one or more processors coupled to the user interface and a memory storing processing instructions. The instructions, when executed by the one or more processors, cause the one or more processors to execute the aforementioned method.

In yet other embodiments, an integrated analyte monitoring assembly includes an analyte sensor designed for whole or partial positioning (i.e., transcutaneous positioning) through a skin layer and for being maintained in in vivo fluid contact with an interstitial fluid (or other) under the skin layer during a predetermined time period, the analyte sensor having a proximal portion and a distal portion, and sensor electronics coupled to the analyte sensor. The sensor electronics include a circuit board having a conductive layer and a sensor antenna disposed on the conductive layer, one or more electrical contacts provided on the circuit board and coupled with the proximal portion of the analyte sensor to maintain continuous electrical communication, and a data processing component provided on the circuit board and in signal communication with the analyte sensor. The data processing component is configured to execute one or more routines for processing signals received from the analyte sensor. The data processing component is configured to detect a radio frequency (RF) power signal, and transmit buffered glucose data and/or real time glucose information generated from an in vivo glucose sensor to a remotely located device, using for example, but not limited to a backscattering radio wave, only when the RF power signal is detected, to perform, using one or more processors, hypoglycemic or impending hypoglycemic condition detection including comparison of a subset of the buffered glucose data to a stored glucose data profile, and confirmation of the hypoglycemic condition based on the comparison, where when the hypoglycemic condition is confirmed, to generate a notification signal and activating a radio frequency (RF) communication module to wirelessly transmit the generated notification signal to the remotely located device, where the RF communication module is only activated when the notification signal is generated, and to update glucose sensor life expiration data each time the notification signal is generated and transmitted such that the sensor life expiration is reduced with each generated notification signal by a predetermined time period.

Numerous other aspects and embodiments are provided. These other features and aspects of the embodiments of the present disclosure will become more fully apparent from the following detailed description, the appended claims, and the accompanying drawings.

### BRIEF DESCRIPTION OF THE DRAWINGS

FIG. 1 depicts a block diagram of the in vivo analyte monitoring system in accordance with some embodiments of the present disclosure;

FIG. 2 is a flowchart illustrating adverse condition notification routine executed in the on body unit sensor electronics in accordance with one embodiment of the present disclosure;

FIG. 3 is a flowchart illustrating adverse condition notification routine executed in the on body unit sensor electronics in accordance with another embodiment of the present disclosure;

FIG. 4 is a flowchart illustrating routine executed in the on body unit sensor electronics to process concurrent occurrence of adverse condition detection and real time glucose level information request in accordance with some embodiments of the present disclosure; and

FIG. 5 is a flowchart illustrating sensor expiration update routine based on adverse condition detection executed in the on body unit sensor electronics in accordance with some embodiments of the present disclosure.

### DETAILED DESCRIPTION

Embodiments of the present disclosure provide methods, apparatuses, assemblies, systems and kits for in vivo monitoring of analyte levels that include on body sensor electronics operatively coupled to an analyte sensor for real time monitoring of analyte levels, where the sensor electronics includes, for example, application specific integrated circuit (ASIC) with programming logic to analyze and process signals from the analyte sensor for detection and generation of adverse condition notification such as detection of an adverse medical condition such as a hypoglycemic condition or impending hypoglycemic condition, based on which, the ASIC is programmed to activate the radio frequency (RF) communication module provided in the sensor electronics of the on body unit to transmit the generated notification to a reader device to inform the user of the detected adverse condition. The analyte sensor may include a transcutaneously positioned in vivo analyte sensor, a transdermal analyte sensor, a fully implantable analyte sensor, an amperometric sensor, a coulometric sensor, an electrochemical sensor, an optical sensor, or the like, which can monitor analyte levels in real time and provide an indication of such monitored analyte level by, for example, generating a corresponding real time signal indicating the monitored analyte level.

Embodiments of the subject disclosure are described primarily with respect to glucose monitoring devices and systems but the described embodiments may be applied to other analytes and analyte characteristics. For example, other analytes that may be monitored include, but are not limited to, acetyl choline, amylase, bilirubin, cholesterol, chorionic gonadotropin, creatine kinase (e.g., CK-MB), creatine, DNA, fructosamine, glutamine, growth hormones, hormones, ketones, lactate, oxygen, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times.

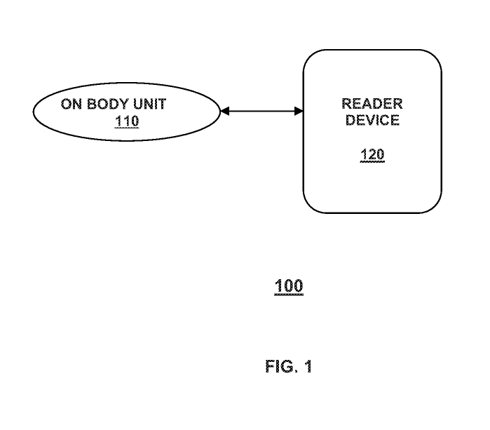

FIG. 1 depicts a block diagram of an in vivo analyte monitoring system in accordance with some embodiments of the present disclosure. Referring to FIG. 1, in one embodiment, the analyte monitoring system 100 includes an on body unit 110 in signal communication with a reader device 120. On body unit 110 is configured for temporary fixed placement on a skin surface of the user, and operatively coupled to a transcutaneously positioned analyte sensor such as an in vivo glucose sensor. Additional details of the analyte monitoring system such as the system 100 described above can be found in U.S. patent applications Ser. Nos. 12/807,278 and 12/698,124, the disclosures of each of which are incorporated herein by reference for all purposes.

Referring to FIG. 1, on body unit 110 in certain embodiments includes hardware such as an ASIC including programming logic to analyze and process the signals (e.g., current signals, voltage signals, or the like) received from the analyte sensor, a memory to store or buffer the analyzed and processed signals in addition to storing parameters for data analysis (e.g., predetermined or programmed hypoglycemic level, and glucose directional variation (e.g., trend) determination algorithm), and a first data communication module such as a radio frequency identification (RFID) module for providing real time glucose data in response to an interrogation signal received from the reader device 120. On body unit 110 in certain embodiments includes a separate adverse event communication module such as a radio frequency (RF) communication module in signal communication with the ASIC for the transmission of the adverse condition detection such that, only when the ASIC determines that a programmed adverse condition is present based on the monitored real time analyte level information received from the in vivo sensor, the adverse event communication module is activated or transitioned from a sleep (low power inactive) state, to activate state for autonomous (relative to the first data communication module) communication of the adverse condition notification or alert to the reader device 120. In certain embodiments, the reader device 120 includes a mobile telephone that supports multiple operation modes including for example, (1) cell phone mode, (2) RF communication mode, and (3) RFID communication mode, for operation as a standalone mobile telephone (supporting cell phone mode), and/or for operation as a communication device (supporting RF and RFID communication modes) in the analyte monitoring system 100 (FIG. 1) for communication with the on body unit 110.

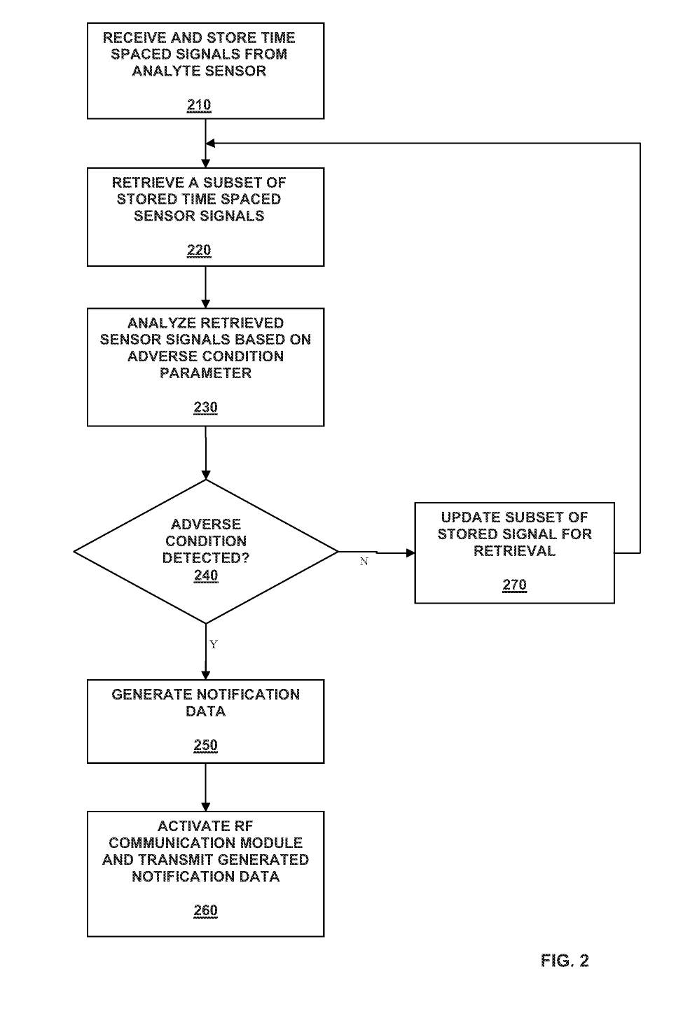

As described in further detail in conjunction with FIGS. 2-5, in certain embodiments, the ASIC of the sensor electronics provided in the on body unit 110 of the analyte monitoring system 100 is configured to process and analyze signals received from the in vivo glucose sensor in real time to determine whether any programmed adverse condition such as hypoglycemic or impending hypoglycemic condition is present based on the analyzed sensor signals, and thereafter, upon determination of the presence of such condition, generates a notification that is then transmitted to the reader device 120. In this manner, the programmed logic in the ASIC of the sensor electronics in the on body unit 110 in certain embodiments autonomously monitors for programmed adverse conditions, and communicates the detected adverse condition to the reader device 120 so that the user or patient can make a timely corrective action. That is, by autonomously monitoring for the programmed adverse condition, the sensor electronics in the on body unit 110 performs sensor data processing to analyze the received sensor data and determine whether such adverse condition is present or not, rather than merely communicating the sensor data to another device located remote to the on body unit 110 (for example, the reader device) for sensor data analysis.

In certain embodiments, the ASIC of the on body unit 110 sensor electronics includes programming logic to update the sensor expiration information with each detection of adverse condition and transmission of corresponding notification. For example, in certain embodiments, the expiration of a sensor may be programmed into the on body unit and/or reader unit to notify a user and/or disable usage after the expiration. In some embodiments, the sensor expiration time period is reduced with each detection of adverse condition and transmission of corresponding notification because each detection of adverse condition and transmission of the adverse condition notification consumes power in the sensor electronics (e.g., the battery provided in the on body unit 110) which is also necessary to power the sensor during the in vivo use period of the sensor in certain embodiments. The ASIC may include programming to reduce an expiration time period by a preset amount each time an adverse condition is detected and the notification is transmitted to the reader device 120. For example, a sensor may have a predetermined expiration period defined as a time starting from sensor manufacture or insertion or initialization of a day, multiple days, a week, multiple weeks, a month, multiple months, a year, or longer. For example, a sensor may have a 14 day useful life starting from sensor insertion or initialization, and the ASIC may include programming to reduce the 14 day in vivo sensor life by a preset amount (such as 1 minute, 5 minutes, 10 minutes, 15 minutes or 30 minutes, for example) each time an adverse condition is detected and a notification is transmitted to the reader device 120.

The various processes described above, including the processes operating in the software application execution environment in the analyte monitoring system, including the sensor electronics of the on body unit and/or the reader device performing one or more routines described above may be embodied as computer programs developed using an object oriented language that allows the modeling of complex systems with modular objects to create abstractions that are representative of real world, physical objects and their interrelationships. The software (e.g., instructions) required to carry out the inventive process, which may be stored in a memory or storage device of the storage unit of the various components of the analyte monitoring system described above in conjunction with the drawing, including the on body unit or the reader device, may be developed by a person of ordinary skill in the art and may include one or more computer program products.

FIG. 2 is a flowchart illustrating adverse condition notification routine executed in the on body unit sensor electronics in accordance with one embodiment of the present disclosure. Referring to FIGS. 1-2, time spaced signals from glucose sensor is received and stored by the sensor electronics (210) under the control of the ASIC programming logic. A subset of the stored time spaced sensor signals is retrieved (220), and the retrieved sensor signals are analyzed in accordance with programmed or stored algorithm(s) by the ASIC in the sensor electronics of the on body unit 110 based on an adverse condition parameter that is stored in sensor electronics (230). For example, in certain embodiments, the ASIC of the sensor electronics is programmed to retrieve a signal received from the sensor indicative of the real time analyte level, and a predetermined number of stored glucose level information based on signals received from the analyte sensor, to determine whether an adverse condition is present (240).

That is, in certain embodiments, the ASIC is programmed to retrieve a predetermined number of sensor signals including the most temporally current sensor signals, each signal obtained at a fixed and/or variable time interval, to determine if at least a subset number of the predetermined number of temporally adjacent sensor signals are below or above a set threshold level indicating a corresponding adverse condition. For example, a most recently generated sensor signal and prior four sensor signals, each signal obtained at one minute intervals, to span a five minute period of monitored glucose level, and to determine if at least three of the five temporally adjacent glucose levels are below the hypoglycemic threshold level (for example, below 60 mg/dL, or some other suitable/desired level stored in the sensor electronics). If the ASIC determines that the at least three of the five temporally adjacent glucose levels is below the hypoglycemic threshold level (that is, the monitored glucose level is below 60 mg/dL for at least three consecutive minutes), the ASIC of the sensor electronics generates a notification data which can be a single data bit representative of an adverse condition alert (250). Then, using an RF communication module of the sensor electronics that is activated by the programming logic in the ASIC, transmits the notification data to the reader device 120 (FIG. 1) over an RF communication link using the RF communication module (260). If the ASIC determines that the analyzed data does not indicate hypoglycemia, then no transmission is activated.

Referring back to FIG. 2, if the adverse condition is not detected (240) based on the analysis of the retrieved subset of the stored time spaced sensor signals, then the subset of stored signals for retrieval is updated (270), and the routine returns to retrieve the stored time spaced sensor signals based on the updated subset. That is, in certain embodiments, the updated subset of sensor signals for retrieval and analysis by the ASIC programming logic may include time shifted consecutive sensor signals (for example, the new sensor signal obtained from the sensor and the prior four stored sensor signals). In other embodiments, the subset of sensor signals for retrieval may be increased or decreased so that more or less sensor signals, respectively, are retrieved for adverse condition analysis.

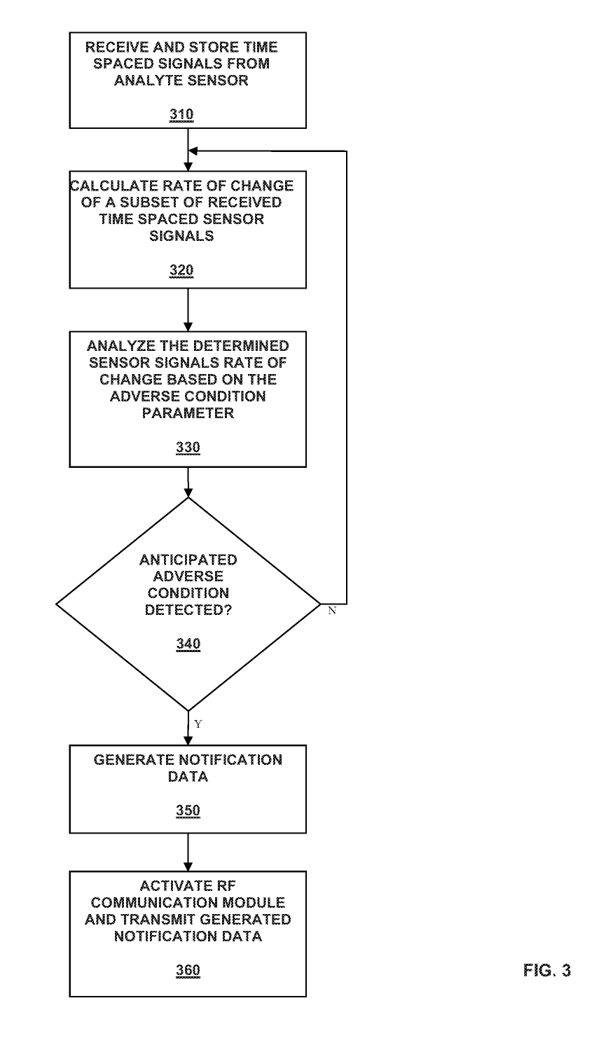

FIG. 3 is a flowchart illustrating adverse condition notification routine executed in the on body unit sensor electronics in accordance with another embodiment of the present disclosure. Referring to FIG. 3, the ASIC programming logic of the sensor electronics in the on body unit 110 (FIG. 1) in one embodiment receives and stores time spaced signals from the transcutaneously positioned analyte sensor such as glucose sensor (310) and, using one or more algorithms stored in the sensor electronics, the ASIC logic calculates a rate of change of the glucose level based on a subset of received and stored time spaced sensor signals (320). The determined rate of change of the glucose level is then analyzed by the ASIC logic based on stored adverse condition parameter (330) to ascertain the detection of an anticipated adverse condition such as an impending hypoglycemic condition based on the rate of change of the glucose level (340). When the anticipated adverse condition is detected, ASIC is programmed to generate a corresponding notification data (350), and to activate the RF communication module of the sensor electronics in the on body unit 110 (FIG. 1) to transmit the generated notification data (360) to the reader device 120.

Referring back to FIG. 3, if the anticipated adverse condition is not detected based on the analysis of the rate of change calculation by the ASIC programming logic based on the adverse condition parameter, the routine returns to retrieve the next subset of time spaced sensor signals to determine a new rate of change of glucose level. In certain embodiments, the adverse condition parameter stored in the sensor electronics of the on body unit 110 may be a threshold rate of −2 mg/dL/min within a preset level from the stored hypoglycemic level (e.g., 60 mg/dL) such that when the current glucose level is at the stored preset glucose level, and the determined rate of change of at −2 mg/dL/min, a projection of the glucose level based on the determined rate of change will result in the glucose level crossing stored hypoglycemic level within a fixed time period. Alternatively, the adverse condition parameter stored in the sensor electronics of the on body unit 110 may be a threshold rate of +2 mg/dL/min within a preset level from the stored hypoglycemic level (e.g., 180 mg/dL) such that when the current glucose level is at the stored preset glucose level, and the determined rate of change of at +2 mg/dL/min, a projection of the glucose level based on the determined rate of change will result in the glucose level crossing stored hypoglycemic level within a fixed time period. While specific numerical examples are provided above, within the scope of the present disclosure, other suitable rates of changes and threshold levels are contemplated such as, for example, +/−2.5 mg/dL/min, +/−3 mg/dL/min; +/−3.5 mg/dL/min, or +/−4 mg/dL/min.

Accordingly, the sensor electronics of the on body unit 110, in certain embodiments, is programmed to generate a notification based on the adverse condition analysis and transmit the notification to the reader device 120 to alert the user or the patient to take necessary corrective actions. Within the scope of the present disclosure, the sensor electronics of the on body unit 110 monitors for other conditions or parameters associated with the on body unit 110 such as, but not limited to the sensor status (failure mode detection), sensor signal/data corruption, sensor dislodging detection, each based on a pre-programmed algorithm provided to the sensor electronics to monitor for and detect operating conditions of the on body unit 110. Upon monitoring and detection of the one or more of such conditions or parameters of the on body unit 110, the sensor electronics of the present disclosure autonomously provides or communicates the detected condition/parameter to the remotely located reader device 120 (FIG. 1) or other suitable data communication device.

In accordance with certain embodiments of the present disclosure, the adverse condition determination by the sensor electronics of the on body unit 110 in the analyte monitoring system 100 (FIG. 1) and subsequent communication of notification to the reader device 120 is independent of the analyte data communication based on RFID communication where the ASIC of the sensor electronics in the on body unit 110 includes RFID module to provide real time analyte level information to the reader device 120 in response to and when the interrogation signal is received from the reader device 120 as determined by the user of the device. In this manner, the ASIC in the sensor electronics of the on body unit 110 is configured to control the operation of the on body unit sensor electronics and process sensor signals such that real time glucose data is provided upon request by the reader device 120, and in addition, autonomously determine adverse conditions based on monitored analyte levels, and to transmit notification to reader device using a separate RF communication module.

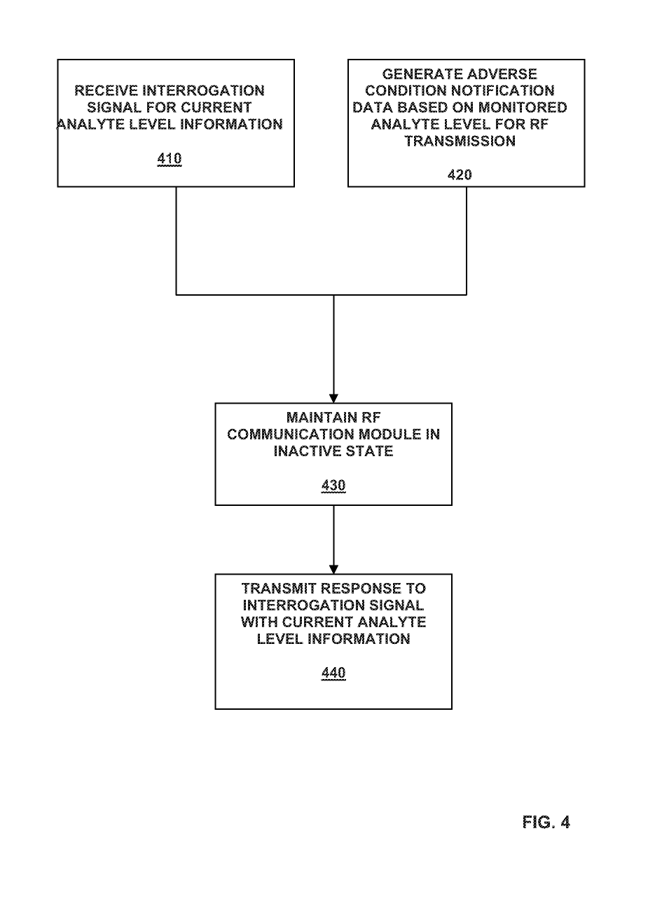

FIG. 4 is a flowchart illustrating routine executed in the on body unit sensor electronics to process a concurrent occurrence of an adverse condition detection and a real time glucose level information request in accordance with some embodiments of the present disclosure. Referring to FIG. 4, in certain embodiments, sensor electronics in the on body unit 110 (FIG. 1) receives an interrogation signal or request from the reader device 120 for current real time and/or buffered/stored analyte level information (410). Concurrently, the sensor electronics of the on body unit 110 generates adverse condition notification data for transmission based on the analysis of the monitored analyte level (420). In certain embodiments, the ASIC programming logic of the sensor electronics in the on body unit 110 is programmed to prioritize between the transmission of the real time analyte level information (RFID communication) and the adverse condition notification (RF communication) by selecting the RFID response data packet to provide to the reader device 120 and suppress the activation of the RF communication module in the on body unit 110 (or maintain the RF communication module in inactive state (430)). In this case, the output of the current analyte level on the reader device 120 based on the communication of the response to the interrogation signal (440) provides the real time analyte level such as glucose level, so that the user will be notified of any adverse or potentially adverse condition based on the real time glucose level.

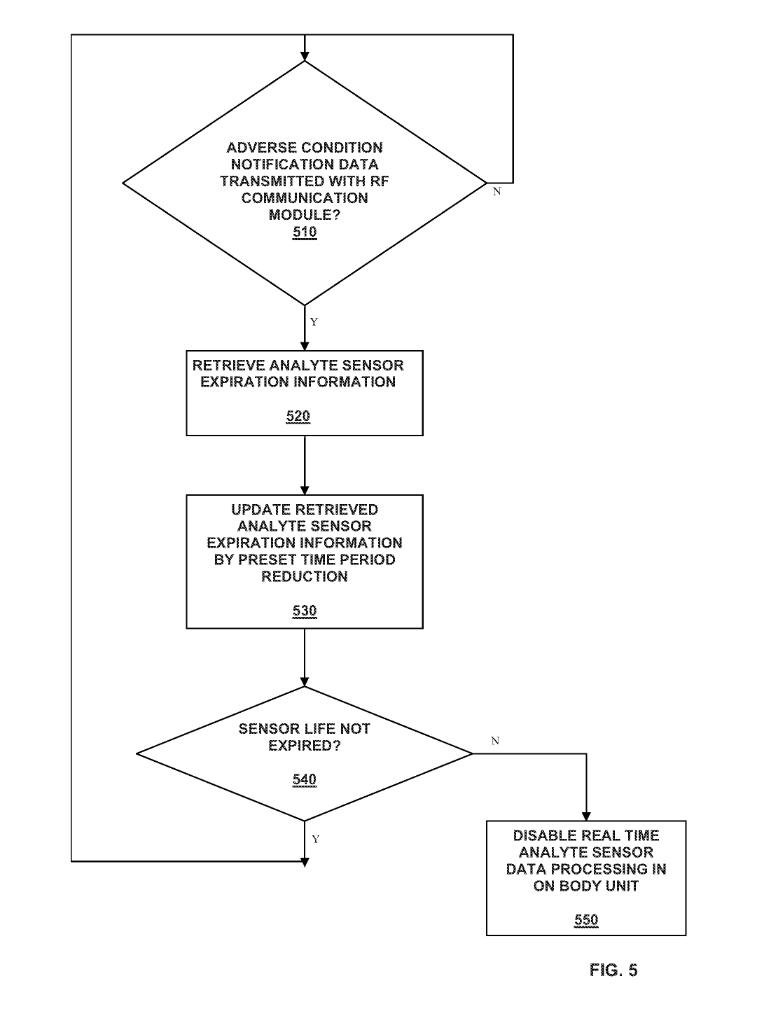

FIG. 5 is a flowchart illustrating sensor expiration update routine based on adverse condition detection executed in the on body unit sensor electronics in accordance with some embodiments of the present disclosure. Referring to FIG. 5, when adverse condition notification data is transmitted with the RF communication module in the sensor electronics of the on body unit 110 (510), ASIC programming logic in certain embodiments retrieves the analyte sensor expiration information from memory or storage device of the sensor electronics (520), and updates the retrieved analyte sensor expiration information by a preset, programmed time period so that the sensor life is effectively reduced upon occurrence of each adverse condition notification data transmission (530). After updating the sensor expiration information, if it is determined that the sensor life has not expired (540), the routine returns to detect whether adverse condition notification data is transmitted (510) as described, for example, in conjunction with FIGS. 2-4 above. On the other hand, if it is determined that the sensor life has expired based on the updated sensor expiration information (540), real time analyte sensor data processing in on body unit 110 by the ASIC programming logic is disabled (550) (and/or the user interface presents an expiration indicator), so that subsequent RFID data packets in response to interrogation signals received from the reader device 120 (FIG. 1) do not include (in other words omit) current, real time monitored analyte level. However, communication of stored data including stored glucose data, temperature data and the like may still be communicated to the reader device 120.

In certain embodiments, sensor electronics of the on body unit 110 is programmed to support scheduled transmission of stored glucose data to the remotely located reader device 120 in the analyte monitoring system 100 (FIG. 1), for example, preceding or following a particular event as a meal event, exercise event, and the like. In certain embodiments, the reader device 120 can program or instruct the sensor electronics of the on body unit 110 with the timing of the scheduled transmission of the stored glucose data. For example, the user, manipulating the operation of the reader device 120 can program the sensor electronics of the on body unit 110 to transmit all or a portion of the stored glucose data before and after each scheduled meal event, or exercise event.

Accordingly, when the programmed event such as the scheduled meal event or exercise event approaches, the sensor electronics of the on body unit 110 retrieves the stored glucose data and transmits to the remotely located reader device. Further, when the scheduled event has been completed (based on, for example, time elapsed from the programmed start of the scheduled event), the sensor electronics of the on body unit 110 retrieves all or a portion of the stored glucose data (or in some cases, the incremental stored glucose data since the last transmission of the glucose data at the beginning of the scheduled event) and again, transmits the retrieved glucose data to the reader device. In this manner, in certain embodiments, a snapshot of glucose profile/measurements can be provided to the user in the context of particular scheduled events based on which, the sensor electronics of the on body unit 110 is programmed for glucose data communication. Within the scope of the present disclosure, other scheduled events are contemplated such as sleep event, travel event, and further, each event can be further segmented, for example, where the meal event can be segmented as breakfast event, lunch event and dinner event, each of which can have a particular amount of time (preceding and/or following) for glucose data transmission from the on body unit 110.

Additionally, in certain embodiments, with each scheduled data communication from the on body unit 110 to the remotely located reader device 120, the on body unit 110 battery life may be monitored and reduced accordingly since each data communication from the on body unit 110 will consume battery power such that the use life of the on body unit 110 may be reduced.

In the manner described above, the analyte monitoring system 100 (FIG. 1) in certain embodiments includes ASIC programming logic in the compact on body unit 110 worn on the body of the user that monitors for adverse conditions based on the glucose signals from the in vivo glucose sensor, and autonomously generates notification which is communicated to the reader device 120 when the adverse condition is detected. The generated notification data in certain embodiments is a single data bit which is communicated over a RF communication link. In addition to the notification data, stored and current glucose data and other related information such as temperature data may be transmitted using the RF communication link. However, in certain embodiments, in the event that the glucose data or other related data such as temperature data, sensor expiration data and the like are corrupted during encryption/decryption or RF communication so that on the reader device 120 the information is not retrievable, the adverse condition notification is still provided on the reader device 120 for output. In still further embodiments, the RFID component and the RF communication module of the on body unit 110 may be configured to share the same antenna that supports RFID communication as well as RF communication with the reader device 120. In other embodiments, two or more separate antennas may be provided in the on body unit 110 to support the RFID data transfer and the RF communication with the reader device 120.

A method in one embodiment includes receiving time spaced glucose signals from an in vivo glucose sensor in fluid contact with interstitial fluid, buffering the received time spaced glucose signals in a memory, detecting a request for real time glucose level information, wherein when the request for real time glucose level information is detected, transmitting the buffered glucose signals and real time glucose signal received from the glucose sensor to a remotely located device using, for example, but not limited to, a backscattering radio wave, processing a subset of the received time spaced glucose signals to identify a predetermined number of consecutive glucose data points from the subset of the received time spaced glucose signals indicating an impending hypoglycemic condition, confirming the impending hypoglycemic condition based on comparison of the predetermined number of consecutive glucose data points to a stored glucose data profile associated with the impending hypoglycemic condition, wherein confirming the impending hypoglycemic condition includes generating a notification signal when the impending hypoglycemic condition is confirmed, activating a radio frequency (RF) communication module to wirelessly transmit the generated notification signal to the remotely located device only when the notification signal is generated.

In certain embodiments, the method includes transmitting the generated notification signal and the time spaced glucose data concurrently, wherein only the generated notification signal is transmitted with the activated RF communication module.

In certain embodiments, when the request for real time glucose level information is detected at the same time as the confirmation of the impending hypoglycemic condition, the method includes prioritizing data transmission such that the generated notification signal is transmitted to the remotely located device after the transmission of the buffered glucose signals and real time glucose signal.

In certain embodiments, wherein when the request for real time glucose level information is detected at the same time as the confirmation of the impending hypoglycemic condition, the method includes suppressing transmission of the generated notification signal such that only the buffered glucose signals and real time glucose signal are transmitted.

In certain embodiments, the method includes updating the glucose sensor expiration information based on the number of generated notification signals during the in vivo use period of the glucose sensor, where updating the glucose expiration information includes subtracting a predetermined amount of time period from the glucose sensor expiration information for each generated notification signal such that the glucose sensor expiration information is shortened with each generated notification signal.

A method in accordance with another embodiment includes detecting a radio frequency (RF) power signal, and transmitting buffered glucose data and real time glucose information generated from an in vivo glucose sensor to a remotely located device using a backscattering radio wave only when the RF power signal is detected, performing, using one or more processors, hypoglycemic condition detection including comparing a subset of the buffered glucose data to a stored glucose data profile, and confirming the hypoglycemic condition based on the comparison, wherein when the hypoglycemic condition is confirmed, generating a notification signal and activating a radio frequency (RF) communication module to wirelessly transmit the generated notification signal to the remotely located device, wherein the RF communication module is only activated when the notification signal is generated, and updating glucose sensor life expiration data each time the notification signal is generated and transmitted such that the sensor life expiration is reduced with each generated notification signal by a predetermined time period.

In certain embodiments, when the RF power signal detection coincides with when the notification signal is generated, the method includes prioritizing data transmission such that the generated notification signal is transmitted to the remotely located device prior to transmitting the buffered glucose data and the real time glucose information.

In certain embodiments, the buffered glucose data and/or real time glucose information are transmitted to the remotely located device using radio frequency identification (RFID) data communication protocol, and the notification signal is transmitted to the remotely located device using RF data communication protocol.

In certain embodiments, the glucose sensor life expiration is subtracted by the predetermined time period with each generated notification signal.

In certain embodiments, the method also includes disabling data communication to the remotely located device when the glucose sensor life has expired.

An apparatus for providing adverse condition notification in an analyte monitoring system in accordance with another embodiment includes an in vivo glucose sensor transcutaneously positioned in fluid contact with interstitial fluid, sensor electronics operatively coupled to the glucose sensor, the sensor electronics including a memory, a radio frequency (RF) communication module, and an application specific integrated circuit (ASIC), the ASIC having programming logic to buffer the received time spaced glucose signals in the memory, to detect a request for real time glucose level information, wherein when the request for real time glucose level information is detected, transmitting the buffered glucose signals and/or real time glucose signal received from the glucose sensor to a remotely located device using, for example, but not limited to, a backscattering radio wave, to process a subset of the received time spaced glucose signals to identify a predetermined number of consecutive glucose data points from the subset of the received time spaced glucose signals indicating an impending hypoglycemic condition, to confirm the impending hypoglycemic condition based on comparison of the predetermined number of consecutive glucose data points to a stored glucose data profile associated with the impending hypoglycemic condition, wherein confirming the impending hypoglycemic condition includes generating a notification signal when the impending hypoglycemic condition is confirmed, and to activate the RF communication module to wirelessly transmit the generated notification signal to the remotely located device only when the notification signal is generated.

In certain embodiments, the ASIC transmits the generated notification signal and the time spaced glucose data concurrently, where only the generated notification signal is transmitted with the activated RF communication module.

In certain embodiments, when the request for real time glucose level information is detected at the same time as the confirmation of the impending hypoglycemic condition, the ASIC prioritizes data transmission such that the generated notification signal is transmitted to the remotely located device after to the transmission of the buffered glucose signals and real time glucose signal.

In certain embodiments, when the request for real time glucose level information is detected at the same time as the confirmation of the impending hypoglycemic condition, the ASIC is programmed to transmit the buffered glucose signals and real time glucose signal and to suppress the communication of the generated notification signal.

In certain embodiments, the ASIC is programmed to update the glucose sensor expiration information based on the number of generated notification signals during the in vivo use period of the glucose sensor, where the ASIC is programmed to subtract a predetermined amount of time period from the glucose sensor expiration information for each generated notification signal such that the glucose sensor expiration information is shortened with each generated notification signal.

A glucose monitoring apparatus in accordance with another embodiment includes an in vivo glucose sensor having a portion transcutaneously positioned in fluid contact with interstitial fluid, sensor electronics including an application specific integrated circuit (ASIC) having programming logic to detect a radio frequency (RF) power signal, and to transmit buffered glucose data and real time glucose information generated from the glucose sensor to a remotely located device using, for example, but not limited to, a backscattering radio wave only when the RF power signal is detected, to perform hypoglycemic condition detection including comparing a subset of the buffered glucose data to a stored glucose data profile, and to confirm the hypoglycemic condition based on the comparison, wherein when the hypoglycemic condition is confirmed, to generate a notification signal and activate a radio frequency (RF) communication module to wirelessly transmit the generated notification signal to the remotely located device, wherein the RF communication module is only activated when the notification signal is generated, wherein the programming logic of the ASIC further includes updating glucose sensor life expiration data each time the notification signal is generated and transmitted such that the sensor life expiration is reduced with each generated notification signal by a predetermined time period.

In certain embodiments, when the RF power signal detection coincides with when the notification signal is generated, the programming logic of ASIC prioritizes data transmission such that the transmission of the generated notification signal to the remotely located device is suppressed so that only the buffered glucose data and the real time glucose information is communicated to the remotely located device.

In certain embodiments, the buffered glucose data and real time glucose information are transmitted to the remotely located device using radio frequency identification (RFID) data communication protocol, and wherein the notification signal is transmitted to the remotely located device using RF data communication protocol.

In certain embodiments, the glucose sensor life expiration is subtracted by the predetermined time period with each generated notification signal.

In certain embodiments, the programming logic of the ASIC disables data communication to the remotely located device when the glucose sensor life has expired.

In accordance with still a further embodiment, there is provided a method of providing physiological data communication, comprising receiving time spaced glucose related signals from an in vivo glucose sensor in fluid contact with interstitial fluid, storing the received time spaced glucose related signals in a memory, detecting a predetermined time remaining to the occurrence of a scheduled programmed event, retrieving at least a portion of the stored received time spaced glucose related signals from the memory, and transmitting the retrieved at least a portion of the stored received time spaced glucose related signals to a remote location.

In certain embodiments, the scheduled programmed event includes one or more of a scheduled meal event, a sleep event, an exercise event, or a travel event.

In certain embodiments, retrieving at least a portion of the stored received time spaced glucose related signals from the memory includes retrieving the entire stored received time spaced glucose related signals from the memory, and further including transmitting the entire retrieved stored received time spaced glucose related signals from the memory to the remote location.

In still further embodiments, detecting the predetermined time remaining to the occurrence of the scheduled programmed event includes monitoring time remaining from the occurrence of the scheduled programmed event.

In yet further embodiments, the method includes determining the frequency of the detection of the predetermined time remaining to the occurrence of a scheduled programmed event, and transmission of the retrieved at least a portion of the stored received time spaced glucose related signals to the remote location, and adjusting the glucose sensor expiration information based on the determined frequency of detection.

Various other modifications and alterations in the structure and method of operation of the embodiments of the present disclosure will be apparent to those skilled in the art without departing from the scope and spirit of the present disclosure. Although the present disclosure has been described in connection with certain embodiments, it should be understood that the present disclosure as claimed should not be unduly limited to such embodiments. It is intended that the following claims define the scope of the present disclosure and that structures and methods within the scope of these claims and their equivalents be covered thereby.

1. (canceled)

2. A method for determining an end of life of a continuous analyte sensor, comprising:
evaluating a plurality of risk factors associated with end of life symptoms of a sensor;
determining an end of life status of the sensor based on the evaluation of the plurality of risk factors; and
providing an output related to the end of life status of the sensor,
wherein the plurality of risk factors comprise at least two risk factors selected from the group consisting of a number of days the sensor has been in use, a rate of change of sensor sensitivity, end of life noise, oxygen concentration, glucose patterns, error between reference values, and sensor values in clinical units, wherein sensor electronics are configured to evaluate a plurality of risk factors by translating outputs of the plurality of risk factor evaluations to end of life risk factor values, wherein the sensor electronics are configured to determine an end of life status by combining the end of life risk factor values into a combined end of life score, and wherein the sensor electronics are configured to determine an end of life status based on the combined end of life score.

3. A method for determining an end of life of a continuous analyte sensor, comprising:
evaluating a plurality of risk factors associated with end of life symptoms of a sensor;
determining an end of life status of the sensor based on the evaluation of the plurality of risk factors; and
providing an output related to the end of life status of the sensor,
wherein the plurality of risk factors comprise at least two risk factors selected from the group consisting of a number of days the sensor has been in use, a rate of change of sensor sensitivity, end of life noise, oxygen concentration, glucose patterns, error between reference values, and sensor values in clinical units, wherein each of the plurality of risk factors is partially indicative of the end of life of the sensor based on a comparison of the risk factor to one or more criteria, and wherein if at least two of the plurality of risk factors are determined to meet the one or more criteria, respectively, then a combination of the at least two variables is indicative of the end of life of the sensor.
